(19) United States
(12) Patent Application Publication
(10) Pub. No.: US 2025/0213119 A1
(43) Pub. Date: Jul. 3, 2025

(54) METHOD AND SYSTEM FOR ENHANCING RF ENERGY DELIVERY DURING THERMOACOUSTIC IMAGING

(71) Applicant: ENDRA Life Sciences Inc., Ann Arbor, MI (US)

(72) Inventor: Christopher Nelson Davis, Ann Arbor, MI (US)

(73) Assignee: ENDRA Life Sciences Inc., Ann Arbor, MI (US)

(21) Appl. No.: 18/401,439

(22) Filed: Dec. 30, 2023

(51) Int. Cl. A61B 5/00

(52) U.S. Cl. CPC A61B 5/0093

(57) ABSTRACT

A method and system for enhancing radio frequency energy delivery to a region of interest is disclosed. The method includes emitting with a radio frequency (RF) applicator comprising a waveguide and a feed probe, one or more RF energy pulses into the region of interest, detecting with an acoustic receiver, at least one multipolar acoustic pressure wave generated in the region of interest in response to the emitted one or more RF energy pulses and processing data of the at least one multipolar acoustic pressure wave to determine a peak-to-peak amplitude thereof, adjusting a direct-current between the waveguide and the feed probe to tune the RF applicator based on the determined peak-to-peak amplitude, and emitting with the tuned RF applicator, one or more RF energy pulses into the region of interest.

### FIELD

The subject disclosure relates to thermoacoustic imaging and in particular to a method and system for enhancing radio frequency (RF) energy delivery during thermoacoustic imaging.

### BACKGROUND

Thermoacoustic imaging is an imaging modality that provides information relating to the thermoelastic properties of tissue. Thermoacoustic imaging uses short pulses of electromagnetic energy, such as radio frequency (RF) pulses, directed into a subject to heat absorbing features within the subject rapidly, which in turn induces acoustic pressure waves that are detected using acoustic receivers such as one or more thermoacoustic or ultrasound transducer arrays. The detected acoustic pressure waves are analyzed through signal processing, and processed for presentation as thermoacoustic images that can be interpreted by an operator.

In order to direct RF pulses into the subject during thermoacoustic imaging, a radio frequency (RF) applicator is coupled to tissue adjacent a region of interest (ROI) within the subject to be imaged. Sub-optimal coupling of the RF applicator to the tissue may cause issues such as inefficient energy transfer, reduced heating rates, reduced signal intensity, non-uniform energy deposition, tissue hotspots, tissue overheating, RF power supply damage, and poor image quality. Factors that lead to sub-optimal coupling of the RF applicator to the tissue include variability in the size of the subject, the size of tissue within the subject, the geometry of tissue within the subject, the composition of tissue within the subject, etc.

Although techniques for coupling an RF applicator to tissue have been considered, improvements are desired. Hence, a need exists for an improved method and system for enhancing radio frequency (RF) energy delivery during thermoacoustic imaging.

### SUMMARY

A method and system for enhancing radio frequency energy delivery to a region of interest is disclosed. The method includes emitting with a radio frequency (RF) applicator comprising a waveguide and a feed probe, one or more RF energy pulses into the region of interest, detecting with an acoustic receiver, at least one multipolar acoustic pressure wave generated in the region of interest in response to the emitted one or more RF energy pulses and processing data of the at least one multipolar acoustic pressure wave to determine a peak-to-peak amplitude thereof, adjusting a direct-current between the waveguide and the feed probe to tune the RF applicator based on the determined peak-to-peak amplitude, and emitting with the tuned RF applicator, one or more RF energy pulses into the region of interest.

In one embodiment, method for enhancing radio frequency energy delivery to a region of interest comprises: (i) emitting with a radio frequency (RF) applicator comprising a waveguide and a feed probe, one or more RF energy pulses into the region of interest, the region of interest comprising an object of interest and at least one reference that are separated by at least one boundary; (ii) detecting with an acoustic receiver, at least one multipolar acoustic pressure wave generated in the region of interest in response to the emitted one or more RF energy pulses and processing data of the at least one multipolar acoustic pressure wave to determine a peak-to-peak amplitude thereof; (iii) adjusting a direct-current between the waveguide and the feed probe to tune the RF applicator based on the determined peak-to-peak amplitude, the adjustment selected to maximize the peak-to-peak amplitude; and (iv) emitting with the tuned RF applicator, one or more RF energy pulses into the region of interest.

In one embodiment, a system for enhancing radio frequency energy delivery to a region of interest, comprising an object of interest and a reference that are separated by at least one boundary, comprises: a thermoacoustic imaging system comprising a tunable radio frequency (RF) applicator which includes a waveguide and a feed probe configured to emit RF energy pulses into the region of interest to heat the object of interest and the reference that are separated by at least one boundary, means to adjust a direct-current between the waveguide and the feed probe, and an acoustic receiver configured to receive multipolar acoustic pressure waves generated in response to the heating of the object of interest and the reference that are separated by at least one boundary, wherein the multipolar acoustic pressure waves are generated in the region of interest at the boundary; and one or more processors configured to: process the received multipolar acoustic pressure waves to determine a tuning setting for the RF applicator that yields, in response to the adjustment of the direct-current between the waveguide and the feed probe, multipolar acoustic pressure waves with maximum peak-to-peak amplitudes in the region of interest.

In one embodiment, the method further comprises performing thermoacoustic imaging of the region of interest using the tuned RF applicator.

In one embodiment, the method further comprises determining one or more parameters of the object of interest from the thermoacoustic imaging.

In one embodiment, the one or more parameters of the object of interest comprise at least one of fractional fat content and temperature.

In one embodiment, the region of interest comprises tissue and further wherein the object of interest and the at least one reference are different types of tissue.

In one embodiment, the different types of tissue are selected from a group consisting of muscle tissue, fat tissue, blood vessel tissue, liver tissue, and kidney tissue.

This summary is provided merely to introduce certain concepts and not to identify key or essential features of the claimed subject matter.

### BRIEF DESCRIPTION OF THE DRAWINGS

Embodiments will now be described more fully with reference to the accompanying drawings in which:

FIG. 1 is a schematic view of an exemplary imaging system;

FIGS. 2-13 depict an exemplary radio frequency (RF) applicator forming part of the imaging system of FIG. 1;

FIG. 14 is a cross-sectional view of the RF applicator;

FIG. 15 is a graph showing exemplary multipolar acoustic signals;

FIG. 16 is a flow chart depicting exemplary steps for enhancing radio frequency delivery during thermoacoustic imaging;

FIG. 17 depicts an exemplary multipolar acoustic signal obtained using the steps of FIG. 16; and

FIG. 18 shows various parts of a human body that can be imaged using the teachings herein; and

FIG. 19 is an exemplary block diagram illustrating an example computing system that can be used in conjunction with one or more of the embodiments of the disclosure.

### DETAILED DESCRIPTION OF THE EMBODIMENTS

The foregoing summary, as well as the following detailed description of certain examples will be better understood when read in conjunction with the appended drawings. As used herein, an element or feature introduced in the singular and preceded by the word “a” or “an” should be understood as not necessarily excluding the plural of the elements or features. Further, references to “one example” or “one embodiment” are not intended to be interpreted as excluding the existence of additional examples or embodiments that also incorporate the described elements or features. Moreover, unless explicitly stated to the contrary, examples or embodiments “comprising” or “having” or “including” an element or feature or a plurality of elements or features having a particular property may include additional elements or features not having that property. Also, it will be appreciated that the terms “comprises”, “has”, “includes” means “including but not limited to” and the terms “comprising”, “having” and “including” have equivalent meanings.

As used herein, the term “and/or” can include any and all combinations of one or more of the associated listed elements or features.

It will be understood that when an element or feature is referred to as being “on”, “attached” to, “connected” to, “coupled” with, “contacting”, etc. another element or feature, that element or feature can be directly on, attached to, connected to, coupled with or contacting the other element or feature or intervening elements may also be present. In contrast, when an element or feature is referred to as being, for example, “directly on”, “directly attached” to, “directly connected” to, “directly coupled” with or “directly contacting” another element of feature, there are no intervening elements or features present.

It will be understood that spatially relative terms, such as “under”, “below”, “lower”, “over”, “above”, “upper”, “front”, “back” and the like, may be used herein for ease of description to describe the relationship of an element or feature to another element or feature as illustrated in the figures. The spatially relative terms can however, encompass different orientations in use or operation in addition to the orientations depicted in the figures.

In the following, a method and system for enhancing radio frequency (RF) energy delivery during thermoacoustic imaging are described.

In one embodiment, thermoacoustic imaging is utilized to determine a material type of an object of interest as described in U.S. Pat. No. 11,266,314 (which is herein incorporated by reference in its entirety).

In one embodiment, thermoacoustic imaging is utilized to determine at least one parameter of interest of a material as described in U.S. Pat. No. 11,047,832 (which is herein incorporated by reference in its entirety).

In one embodiment, thermoacoustic imaging is utilized to determine at least one parameter of interest of a material as described in U.S. Pat. No. 11,067,543 (which is herein incorporated by reference in its entirety).

In one embodiment, the method and system utilize an RF applicator to obtain thermoacoustic data of tissue within a region of interest (ROI) of a subject (person or animal). The thermoacoustic data is analyzed and the RF applicator is adjusted to enhance energy delivery to the tissue.

U.S. Pat. No. 11,304,606 (referred to herein as patent '606 and incorporated by reference in its entirety) describes a method and system to enhance radio frequency energy delivery to a region of interest. The present disclosure utilizes different structural embodiments of the RF applicator to better achieve the goals cited in patent '606.

To maximize the RF energy that is coupled to a subject or item that contains an object of interest that is located in a region of interest, the RF energy reflected power must be minimized. So, the antenna (waveguide) resonance frequency must be optimized for the desired frequency of operation. In one embodiment, the desired frequency of operation is 434 MHz. In a separate embodiment, the desired frequency of operation is 915 MHz. Hence, the present disclosure describes different structural embodiments of the RF applicator that optimize the resonance frequency of the RF applicator antenna (waveguide) to minimize reflected power (therefore delivering a maximum amount of energy to the region of interest) at a desired frequency of operation (such as, but not limited to, 434 MHz or 915 MHz).

In actual applications, the impedance (reflected power is a function of impedance) of the antenna can change depending on the tissue composition to which the antenna is coupled. For example, the impedance can change depending on skin moisture and salinity, fat thickness under the skin, and temperature of the antenna. So, the RF applicator may need to be optimized in a dynamic situation where the circumstances are changing during operation.

In addition, manufacturing variations of the antenna components can cause difficulty in achieving a good match for the antenna over a specific frequency bandwidth depending on the tolerance stack up of each antenna component.

The present disclosure describes an RF applicator enabled to adjust the resonance frequency of emitted energy in order to minimize reflected power and conversely maximize the amount of energy transmitted (i.e. optimize permittivity) to the region of interest. In one embodiment, the RF applicator comprises a waveguide, feed probe, and is configured to adjust a direct-current between the waveguide and the feed probe. Adjusting a direct-current between the waveguide and the feed probe enables a change in overall RF applicator permittivity.

The direct-current is used to place a positive charge at a first part of the RF applicator (e.g. the feed probe) and a negative charge at a second part of the RF applicator (e.g. the waveguide). The first part of the RF applicator and the second part of the RF applicator are electrically isolated. Any dielectric material that is between the first part of the RF applicator and the second part of the RF applicator experiences a capacitive effect (i.e. the dielectric material becomes a capacitor) via a change in the dielectric material's permittivity (permittivity is a material property that can be changed), which affects the RF applicator's resonance frequency of emitted energy. Hence, the RF applicator's resonance frequency of emitted energy is a function of the amount of applied direct-current.

In one embodiment, the dielectric material is a coating that is used to coat a block (preferably of ceramic construction) which is an element inserted (residing) in the waveguide. In one embodiment, the coating material of construction is at least partially barium strontium titanate. U.S. Pat. No. 10,682,059 (herein patent '059) describes a coating (wax) that is used to coat a block inserted into a RF applicator waveguide. Patent '059 is incorporated by reference in its entirety.

In one embodiment, the dielectric material is an insert (such as a disc) that is located between the block and waveguide. The feed probe may or may not be touching the insert.

In one embodiment, the dielectric material is a plurality of inserts (such as disks) that are located between the block and waveguide.

In one embodiment, the dielectric material is some combination a coating and one or more inserts.

The permittivity of the dielectric material changes the resonance frequency of the antenna in the following fashion: higher permittivity dielectric material moves the resonance frequency lower while lower permittivity dielectric material moves the resonance frequency higher.

In one embodiment, the antenna can be dynamically tuned in real-time using a bias tee to account for patient, temperature, and manufacturing variation.

Besides using the gap fill as the dielectric material, some to all of the ceramic insert (block) can be replaced with a separate dielectric material and the resonance frequency of the antenna can be changed by applying DC voltage to that section of the ceramic insert (block).

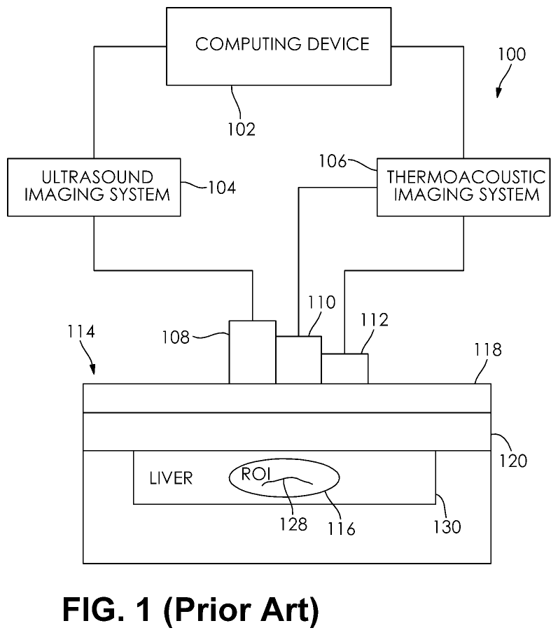

Referring now to the drawings, wherein the depictions are for the purpose of illustrating certain exemplary embodiments only and not for the purpose of limiting the same, FIG. 1 schematically shows an exemplary imaging system 100 is shown and is generally identified by reference numeral 100. As can be seen, the imaging system 100 includes a programmed computing device 102 communicatively coupled to an ultrasound imaging system 104 and to a thermoacoustic imaging system 106. The ultrasound imaging system 104 and thermoacoustic imaging system 106 are configured to obtain ultrasound image data and thermoacoustic image data, respectively, of a region of interest 116. Components of the system 100 are shown in FIG. 1 as single elements. Such illustration is for ease of description, and it should be recognized that the system 100 may include multiple additional imaging devices or sub-devices.

The programmed computing device 102 may be a computer, server or other suitable processing device comprising, for example, a processing unit comprising one or more processors, computer-readable system memory (volatile and/or non-volatile memory), other non-removable or removable computer-readable memory (e.g., a hard disk drive, RAM, ROM, EEPROM, CD-ROM, DVD, flash memory, etc.) and a system bus coupling the various computer components to the processing unit. The computing device 102 may also comprise networking capabilities using Ethernet, Wi-Fi, and/or other suitable network format, to enable connection to shared or remote drives, one or more networked computers, or other networked devices. One or more input devices, such as a mouse and a keyboard (not shown) can be coupled to the computing device 102 for receiving operator input. A display device (not shown), such as one or more computer screens or monitors, can be coupled to the computing device 102 for displaying one or more generated images that are based on ultrasound image data received from the ultrasound imaging system 104 and/or the thermoacoustic image data received from thermoacoustic imaging system 106. The programmed computing device 102 executes program code stored on the computer-readable system memory and/or other non-removable or removable computer-readable memory and performs methods according to the program code as will be described further below.

The ultrasound imaging system 104 comprises an acoustic receiver in the form of an ultrasound transducer 108 that houses one or more ultrasound transducer arrays configured to emit sound waves into the region of interest 116. Sound waves directed into the region of interest 116 echo off materials within the region of interest ROI, with different materials reflecting varying degrees of sound. Echoes that are received by the one or more ultrasound transducer arrays of the ultrasound transducer 108 may be processed by the ultrasound imaging system 104 before being communicated as ultrasound image data to the computing device 102 for further processing and for presentation on the display device as ultrasound images that can be interpreted by an operator. In one embodiment, the ultrasound imaging system 104 utilizes B-mode ultrasound imaging techniques assuming a nominal speed of sound of 1,540 m/s. As ultrasound imaging systems are known in the art, further specifics of the ultrasound imaging system 104 will not be described further herein.

The thermoacoustic imaging system 106 comprises an acoustic receiver in the form of a thermoacoustic transducer 110. The thermoacoustic transducer 110 houses one or more thermoacoustic transducer arrays. Radio-frequency (RF) applicator 112 may be housed together or separately from the thermoacoustic transducer 110. The RF applicator 112 is configured to emit short pulses of RF energy that are directed into the region of interest ROI, which can contain a blood vessel 128. In one embodiment, the RF applicator 112 has a frequency between about 10 Mhz and 100 GHz and has a pulse duration between about 0.1 nanoseconds and 10 microseconds. RF energy pulses delivered to materials within the region of interest 116 induce acoustic pressure waves (thermoacoustic multi-polar signals) within the region of interest 116 that are detected by the thermoacoustic transducer 110. Acoustic pressure waves that are detected by the thermoacoustic transducer 110 are processed and communicated as thermoacoustic image data to the computing device 102 for further processing and for presentation on the display device as thermoacoustic images that can be viewed by the operator.

The coordinate system of the one or more ultrasound transducer arrays of the ultrasound transducer 108 and the coordinate system of the one or more thermoacoustic transducer arrays of the thermoacoustic transducer 110 are mapped by the computing device 102 so that acquired ultrasound and thermoacoustic images can be registered. Alternatively, the thermoacoustic imaging system 106 may make use of the one or more ultrasound transducer arrays of the ultrasound transducer 108 by disconnecting the one or more ultrasound transducer arrays from the ultrasound transducer 108 and connecting the one or more ultrasound transducer arrays to the thermoacoustic transducer 110. As will be appreciated, by doing this coordinate mapping between the one or more ultrasound transducer arrays and the one or more thermoacoustic transducer arrays is not required.

In one embodiment (shown in FIG. 1), an exemplary region of interest 116 contains a blood vessel 128 and is located within a liver 130 of a human or animal body (patient) 114. Patient 114 comprises a skin and subcutaneous fat layer 118 and muscle layer 120 adjacent to liver 130. In a separate embodiment, blood vessel 128 functions as a reference and liver tissue located with the region of interest 116 functions as an object of interest.

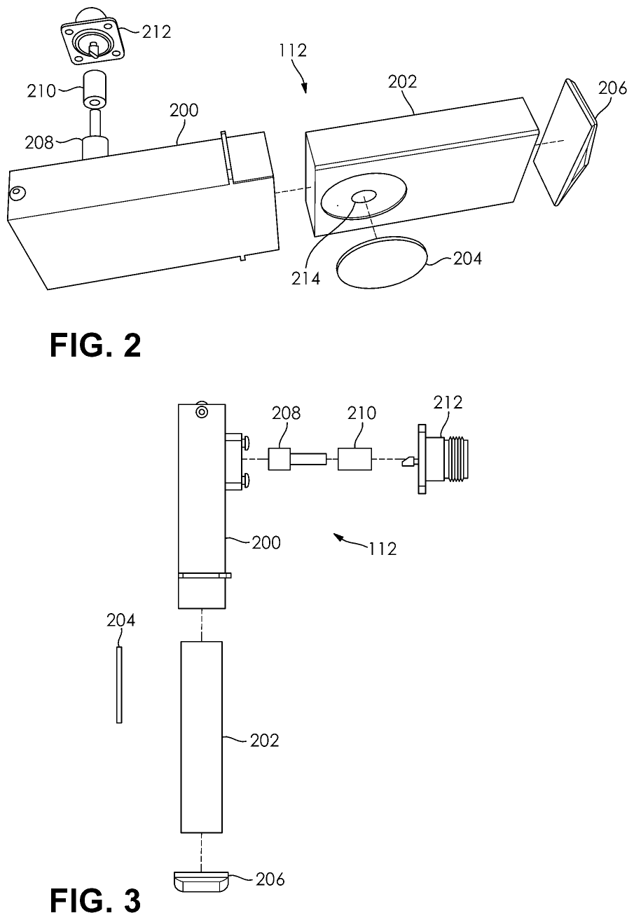

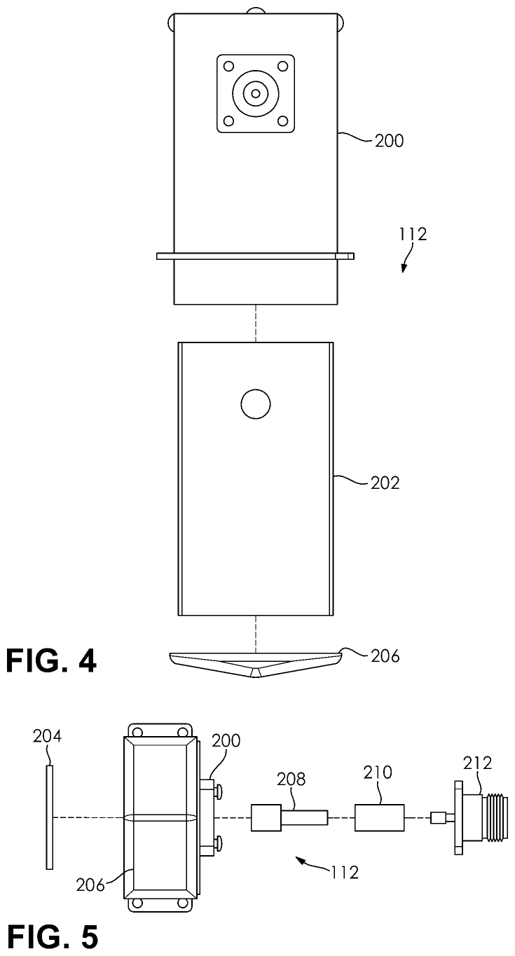

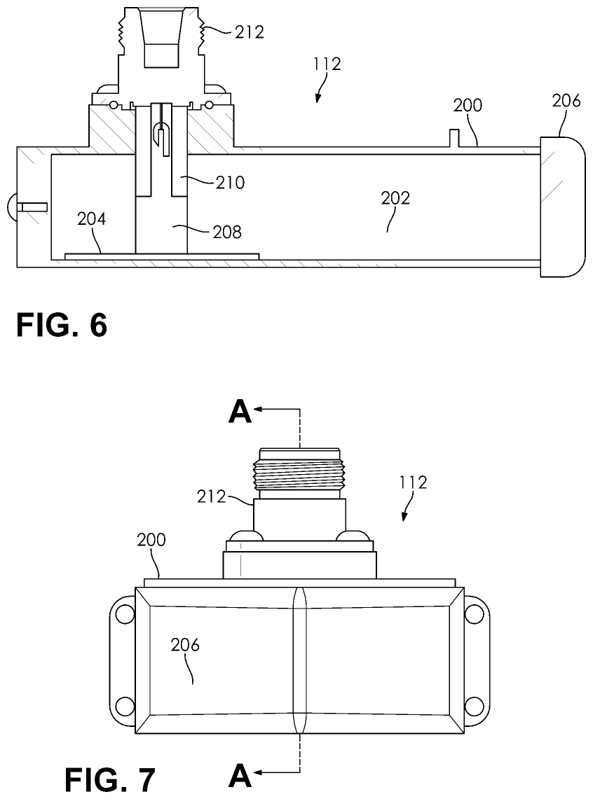

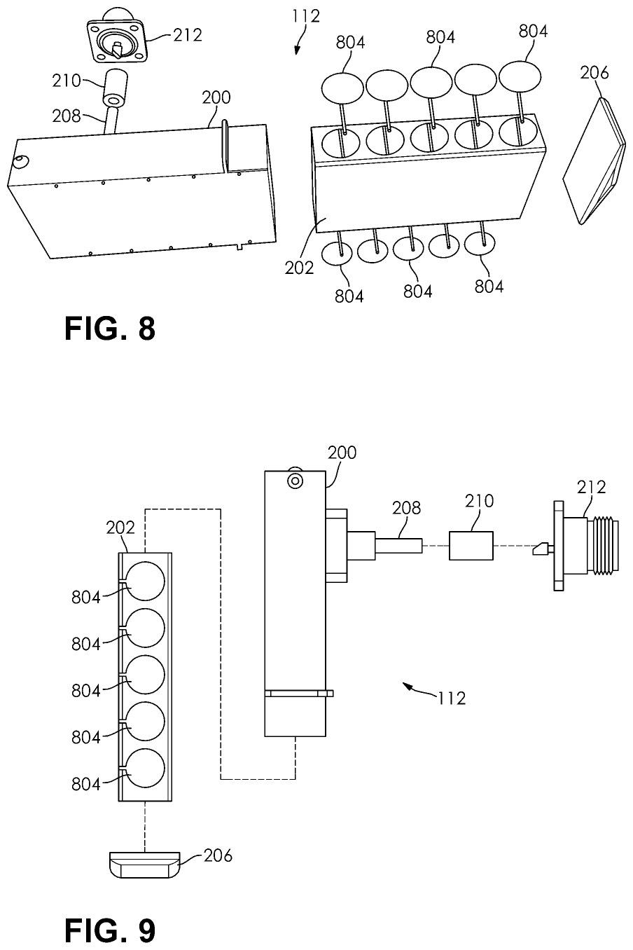

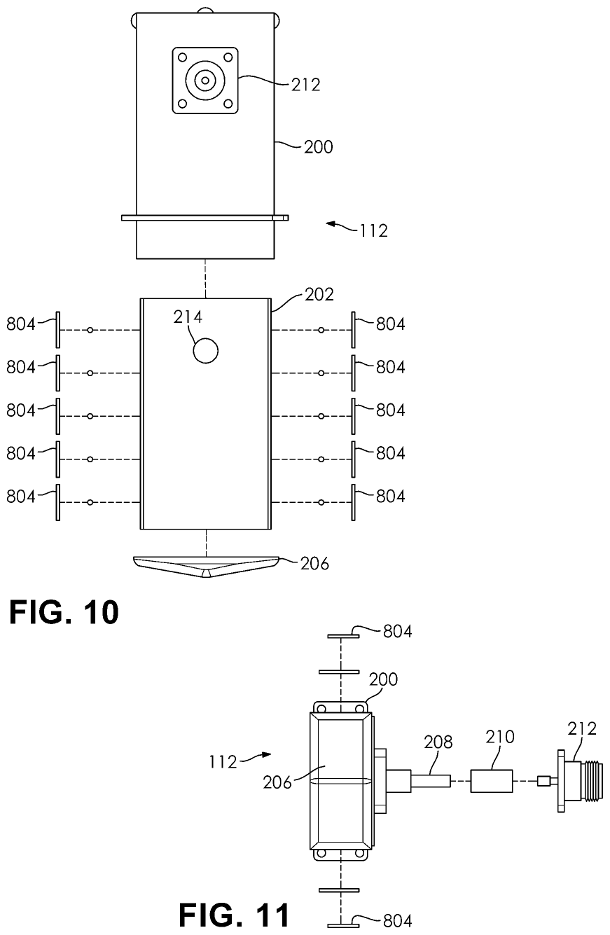

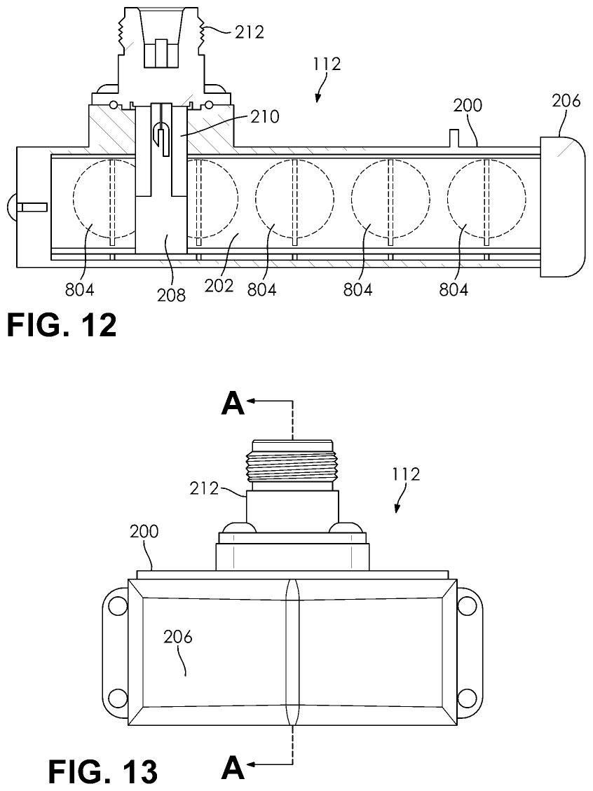

Turning now to FIGS. 2-13, embodiments of the RF applicator 112 are better illustrated. As can be seen, the RF applicator 112 comprises a hollow, generally rectangular, open-ended waveguide (housing) 200 formed of electrically conductive material. A block 202 formed of a high dielectric material lines the interior surface of the hollow open-ended waveguide 200. In one embodiment, block 202 has a real relative permittivity between about 20 and 180 and an imaginary relative permittivity between about 0 and 18. In a separate embodiment, block 202 has a dielectric constant between about 20 and 180.

Also shown in FIGS. 2-13 are primary dielectric insert 204, wedge 206, feed probe 208, insulator 210, connector 212, partially enclosed space 214, and a plurality of secondary dielectric inserts 804.

In one embodiment, the primary dielectric insert 204 material of construction enables greater variability in the real relative permittivity, imaginary relative permittivity, and dielectric constant than the corresponding block 202 parameters when adjusting a direct-current between the open-ended waveguide 200 and the feed probe 208 to tune the RF applicator 112 based on a determined peak-to-peak amplitude.

In one embodiment, the plurality of secondary dielectric inserts 804 material of construction enables greater variability in the real relative permittivity, imaginary relative permittivity, and dielectric constant than the corresponding block 202 parameters when adjusting a direct-current between the open-ended waveguide 200 and the feed probe 208 to tune the RF applicator 112 based on a determined peak-to-peak amplitude.

The feed probe 208, which is configured to generate RF energy pulses, extends through aligned holes in the housing 200 and block 202 so that the feed probe 208 is suspended within the partially enclosed space 214 of the block 202. The feed probe 208 is electrically isolated via insulator 210 and connected to connector 212. The connector 212 enables communication with thermoacoustic imaging system 106. RF energy travels from feed probe 208, through the block 202 and a wedge 206, and into the region of interest ROI.

The wedge 206 is configured to minimize acoustic interference from the RF applicator 112 while maximizing energy delivery to the region of interest 116.

During operation of the RF applicator 112, the frequency of RF energy pulses can be varied by applying a DC voltage across the feed probe 208 and hollow open-ended waveguide 200. In one embodiment, a RF cable 215 that transmits pulses from a source on the thermoacoustic imaging system 106 to the applicator 112 can also carry a DC voltage to tune the applicator. For example, a Bias-tee RF component 213 may be used to add DC to the AC pulse.

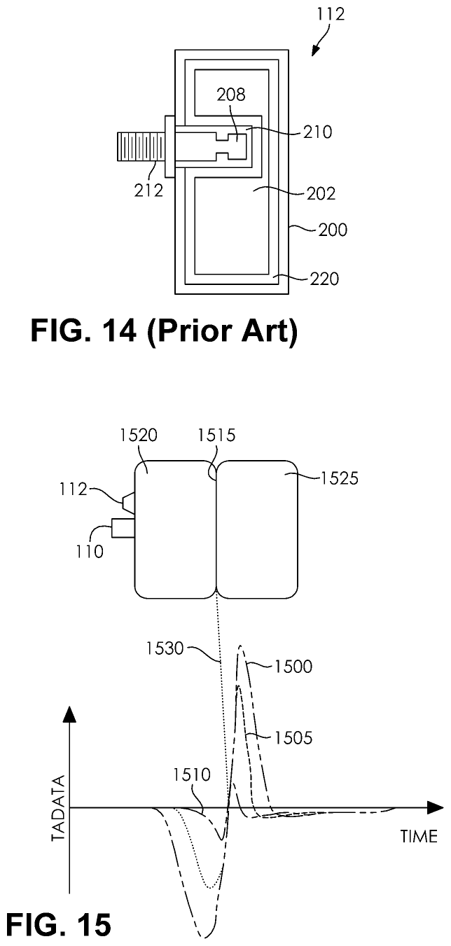

In one embodiment shown in FIG. 14, a coating 220 is used to coat the block 202 to eliminate air gaps between the block 202 and open-ended waveguide (housing) 200. The coating 220 functions as a variable dielectric material. In one embodiment, coating 220 comprises barium strontium titanate or the like. In one embodiment, the coating 220 has a real relative permittivity between about 20 and 180 and an imaginary relative permittivity between about 0 and 18. In a separate embodiment, the coating 220 has a dielectric constant between about 20 and 180. In one embodiment, the coating 220 material of construction enables greater variability in the real relative permittivity, imaginary relative permittivity, and dielectric constant than the corresponding block 202 parameters when adjusting a direct-current between the open-ended waveguide 200 and the feed probe 208 to tune the RF applicator 112 based on a determined peak-to-peak amplitude. FIG. 14 corresponds to FIG. 7 from U.S. Pat. No. 10,682,059, which is herein incorporated by reference in its entirety.

Thermoacoustic imaging can be used to contrast fat or fatty tissues with soft or lean tissues due to their lower electrical conductivity and permittivity in RF compared to other water and ion-rich soft or lean tissues. Fat and fatty tissues also have a lower absorption coefficient compared to soft or lean tissues like muscle. As such, during thermoacoustic imaging of a region of interest that includes a boundary between fat or fatty tissue and soft or lean tissue, multipolar acoustic signals are generated that are received by the thermoacoustic transducer 110. This is due to the fact that the soft or lean tissue absorbs more heat than the fat or fatty tissue causing it to expand rapidly across the boundary and into the fat or fatty tissue, that expands less, and then quickly contract. The strength or peak-to-peak values of the multipolar acoustic signals depend on the relative absorption properties of the fat or fatty tissue and the soft or lean tissue.

FIG. 15 graphically illustrates exemplary multipolar acoustic signals 1500, 1505, and 1510 generated in response to thermoacoustic imaging of an exemplary tissue ROI. The ROI includes a first tissue 1520 and a different type of second tissue 1525 that are separated by a boundary 1515. The dashed line 1530 indicates a time point corresponding to the boundary 1515. The peak-to-peak amplitude of each multipolar acoustic signal 1500, 1505, and 1510 is proportional to a difference in the absorption coefficients of the first tissue 1520 and second tissue 1525. In FIG. 15, the first tissue 1520 is a kidney and has no fat. For multipolar acoustic signal 1500, the second tissue 1525 is a fatty liver that has a high fractional fat content. For multipolar acoustic signal 1505, the second tissue 1525 is an unhealthy liver that has a medium fractional fat content. For multipolar acoustic signal 1510, the second tissue 1525 is a healthy liver that has a low fractional fat content. As can be seen, the peak-to-peak value of multipolar acoustic signal 1500 is greater than that of multipolar acoustic signals 1505, 1510, and the peak-to-peak value of multipolar acoustic signal 1505 is greater than that of multipolar acoustic signal 1510. The differences in the peak-to-peak values of the multipolar acoustic signals 1500, 1505, and 1010 represent a function of the extent to which the first tissue 1520 expands into the boundary 1515 and into the second tissue 1525 before contracting.

Different tissues have characteristic dielectric properties at particular frequencies. The dielectric properties determine how much energy is absorbed by tissue. When RF energy pulses are transmitted through tissue, the RF energy pulses are attenuated. The amount of attenuation can be determined using the dielectric properties of the tissue and the physical properties of the tissue. Fatty tissue absorbs less energy than lean tissue. As such, fatty tissue attenuates the RF energy pulses less than normal tissue. Using these properties, the amount of attenuation of tissue can be estimated and this may be used to determine how much fat is in the tissue. As such, adjusting the frequency of the RF energy pulses emitted by the RF applicator 112 can help to enhance energy delivery during thermoacoustic imaging. In one embodiment, the system 100 is configured to prohibit transmission of RF at certain frequencies. These frequencies may be prohibited for use via government regulations.

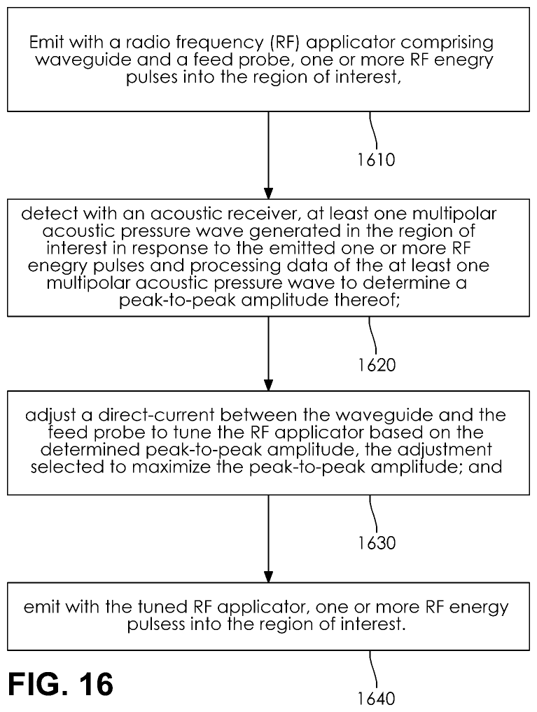

FIG. 16 illustrates an exemplary technique to enhance radio frequency energy delivery to a region of interest using embodiments of the system 100 including the RF applicator 112. The technique is initiated at step 1610, by emitting one or more RF energy pulses into the region of interest 116. The region of interest 116 preferably includes an object of interest (e.g. tissue within a liver) and at least one reference (e.g. a blood vessel within the liver) that are separated by at least one boundary (e.g. an interface between the blood vessel and the liver tissue). At step 1620, the system 100 detects with an acoustic receiver, e.g., the thermoacoustic transducer 110, at least one multipolar acoustic pressure wave generated in the region of interest 116 in response to the emitted one or more RF energy pulses and processing data of the at least one multipolar acoustic pressure wave to determine a peak-to-peak amplitude thereof. At step 1630 the system 100 adjusts a direct-current between the waveguide 200 and the feed probe 208 to tune the RF applicator 112 based on the determined peak-to-peak amplitude, the adjustment selected to maximize the peak-to-peak amplitude. At step 1640, the system 100 emits with the tuned RF applicator 112, one or more RF energy pulses into the region of interest 116.

The schematic flow chart diagrams included herein are generally set forth as logical flow chart diagrams. As such, the depicted order and labeled steps are indicative of one embodiment of the presented process. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the process. For example, an arrow may indicate a waiting or monitoring period of unspecified duration between enumerated steps of the depicted process. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

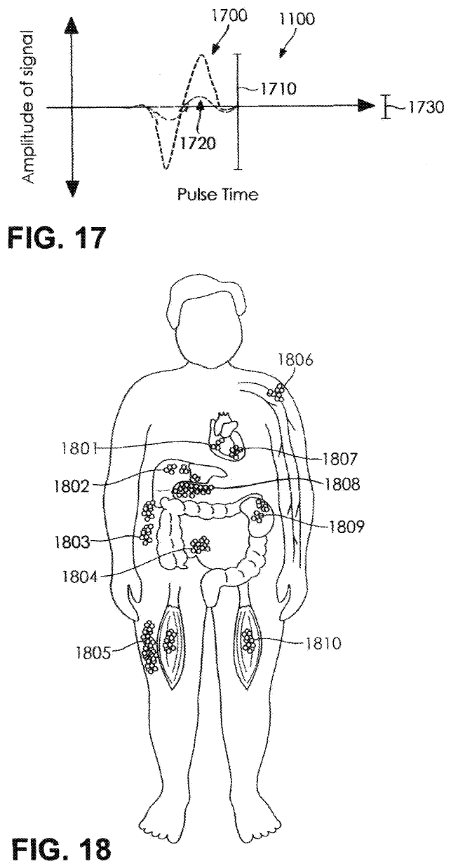

FIG. 17 shows an exemplary multipolar signal 1700 including a peak-to-peak amplitude 1710 that is greater than multipolar signal 1720 peak-to-peak amplitude 1730. An objective of a preferred embodiment is to maximize the peak-to-peak amplitude of the multipolar acoustic pressure wave, enabling more accurate and reliable thermoacoustic imaging. In one embodiment, multipolar signal 1720 is received at step 1620 in response to the emitted RF energy pulse, while multipolar signal 1700 is received after emitting the adjusted RF energy pulses at step 1640.

Although in embodiments the object of interest is described as being the liver and the reference is described as being a blood vessel, those skilled in the art will appreciate that thermoacoustic data may be obtained for other parts of the body. As shown in FIG. 18, various parts of the body that may be imaged using the above-described system and method include the epi/pericardial adipose tissue 1801, the liver 1802, subcutaneous adipose tissue 1803, visceral adipose tissue 1804, subcutaneous gluteal-femoral adipose tissue 1805, perivascular adipose tissue 1806, myocardial fat 1807, pancreas fat 1808, renal sinus fat 1809, and muscle fat 1810.

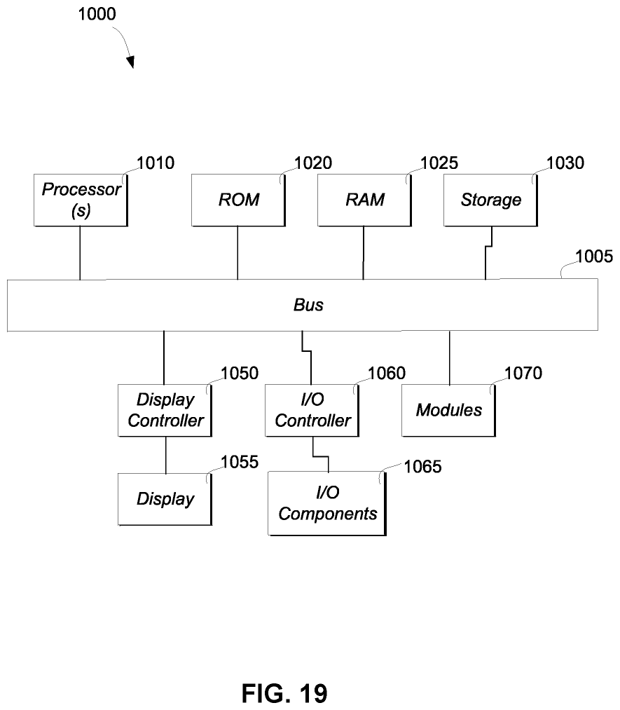

FIG. 19 is an exemplary block diagram illustrating a computing system 1000 that can be used in conjunction with one or more of the embodiments described herein. The illustrated computing system 1000 can represent any of the devices or systems (e.g. the computing device 102, the ultrasound imaging system 104, and/or the thermoacoustic imaging system 106) described herein that perform any of the processes, operations, or methods of the disclosure. Note that while the computing system 1000 illustrates various components, it is not intended to represent any particular architecture or manner of interconnecting the components as such details are not germane to the present disclosure. It will also be appreciated that other types of systems that have fewer or more components than shown may also be used with the present disclosure.

As shown, the computing system 1000 can include a bus 1005 which can be coupled to one or more processors 1010, ROM 1020 (Read Only Memory), RAM 1025 (or volatile memory), and storage 1030 (or non-volatile memory). The processor(s) 1010 can retrieve stored instructions from one or more of the memories (e.g., ROM 1020, RAM 1025, and storage 1030) and execute the instructions to perform processes, operations, or methods described herein. These memories represent examples of a non-transitory machine-readable medium (or computer-readable medium) or storage containing instructions which when executed by a computing system (or a processor), cause the computing system (or processor) to perform operations, processes, or methods described herein. The RAM 1025 can be implemented as, for example, dynamic RAM (DRAM), or other types of memory that require power continually in order to refresh or maintain the data in the memory. Non-volatile memory (e.g., storage 1030) can include, for example, magnetic, semiconductor, tape, optical, removable, non-removable, and other types of storage that maintain data even after power is removed from the system. It should be appreciated that the non-volatile memory can be remote from the system (e.g. accessible via a network).

A display controller 1050 can be coupled to the bus 1005 in order to receive display data to be displayed on a display device 1055, which can display any one of the user interface features or embodiments described herein and can be a local or a remote display device. The computing system 1000 can also include one or more input/output (I/O) components 1065 including mice, keyboards, touch screen, network interfaces, printers, speakers, and other devices. Typically, the input/output components 1065 are coupled to the system through an input/output controller 1060.

Modules 1070 can represent any of the functions or engines described above, including components, units, functions, or logic. Modules 1070 can reside, completely or at least partially, within the memories described above, or within a processor during execution thereof by the computing system. In addition, modules 1070 can be implemented as software, firmware, or functional circuitry within the computing system, or as combinations thereof. In some embodiments, the functions described herein can utilize specialized hardware circuitry (or firmware) of the system.

The various features of the different embodiments or examples may be variously combined with some features included and others excluded to suit a variety of different applications. Examples may include subject matter such as a method, means for performing acts of the method, at least one machine-readable medium including instructions that, when performed by a machine cause the machine to perform acts of the method, or of an apparatus or system according to embodiments and examples described herein. Additionally, various components described herein can be a means for performing the operations or functions described herein.

One embodiment provides an electronic device, comprising a non-transitory machine-readable medium to store instructions; one or more processors to execute the instructions; and a memory coupled to the one or more processors, the memory to store the instructions which, when executed by the one or more processors, cause the one or more processors to detect with an acoustic receiver, at least one multipolar acoustic pressure wave generated in the region of interest in response to the emitted one or more RF energy pulses, process the detected multipolar acoustic pressure wave to determine a peak-to-peak amplitude thereof, identify a tissue type of the region of interest based upon the determined peak-to-peak amplitude, compare the determined peak-to-peak amplitude with an expected peak-to-peak amplitude based upon the identified tissue type to determine attenuation of the RF signal by the region of interest, adjust a frequency of the radio frequency emitted by the RF applicator based upon the determined attenuation, and cause the RF applicator to emit one or more RF energy pulses into the region of interest at the adjusted frequency.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

What is claimed is:

1. A method for enhancing radio frequency energy delivery to a region of interest having an object of interest, the method comprising:
emitting with a radio frequency (RF) applicator comprising a waveguide and a feed probe, one or more RF energy pulses into the region of interest;
detecting with an acoustic receiver, at least one multipolar acoustic pressure wave generated in the region of interest in response to the emitted one or more RF energy pulses and processing data of the at least one multipolar acoustic pressure wave to determine a peak-to-peak amplitude thereof,
adjusting a direct-current between the waveguide and the feed probe to tune the RF applicator based on the determined peak-to-peak amplitude; and
emitting with the tuned RF applicator, one or more RF energy pulses into the region of interest.

2. The method of claim 1, further comprising:
comparing the determined peak-to-peak amplitude with predefined data ranges, the predefined data peak-to-peak ranges, each range associated with a predefined RF energy pulse frequency.

3. The method of claim 2, wherein the adjusting a direct-current between the waveguide and the feed probe to tune the RF applicator based on the determined peak-to-peak amplitude is adjusted based upon the comparing.

4. The method of claim 1, further comprising:
comparing the determined peak-to-peak amplitude with predefined peak-to-peak data ranges, to determine a tissue type;
comparing the determined peak-to-peak amplitude with a predefined amplitude associated with the determined tissue type to determine signal attenuation;
determining a fat content based upon the determined signal attenuation; and
wherein the adjusting the direct-current between the waveguide and the feed probe to tune the RF applicator based on the determined peak-to-peak amplitude is further determined based upon the determined fat content.

5. The method of claim 1, further comprising performing thermoacoustic imaging of the region of interest using the tuned RF applicator.

6. The method of claim 5, further comprising determining one or more parameters of the object of interest from the thermoacoustic imaging.

7. The method of claim 6, wherein the one or more parameters of the object of interest comprise at least one of fractional fat content and temperature.

8. The method of claim 7, wherein the region of interest comprises tissue and further wherein the object of interest comprises at least one reference, and wherein the at least one reference and the object of interest are different types of tissue.

9. The method of claim 8, wherein the different types of tissue are selected from a group consisting of muscle tissue, fat tissue, blood vessel tissue, liver tissue, and kidney tissue.

10. A method for enhancing radio frequency energy delivery to a region of interest having an object of interest, the method comprising:
emitting with a radio frequency (RF) applicator comprising a waveguide and a feed probe, one or more RF energy pulses into the region of interest;
detecting with an acoustic receiver, at least one multipolar acoustic pressure wave generated in the region of interest in response to the emitted one or more RF energy pulses;
processing the detected multipolar acoustic pressure wave to determine a peak-to-peak amplitude thereof;
identifying a tissue type of the region of interest based upon the determined peak-to-peak amplitude;
comparing the determined peak-to-peak amplitude with an expected peak-to-peak amplitude based upon the identified tissue type to determine attenuation of the RF signal by the region of interest;
adjusting a frequency of the radio frequency emitted based upon the determined attenuation; and
emitting with the RF applicator, one or more RF energy pulses into the region of interest at the adjusted frequency.

11. The method of claim 10, wherein the adjusting the frequency of the radio frequency emitted based upon the determined attenuation further includes:
comparing the determined attenuation with a predefined range of attenuation data ranges, wherein each predefined range of attenuation data ranges are associated with a frequency.

10. The method of claim 10, wherein the identifying the tissue type of the region of interest is determined by comparing the determined peak-to-peak amplitude with a table of predefined data ranges, each data range associated with a tissue type.

13. The method of claim 10, wherein each tissue type is associated with an expected peak-to-peak amplitude.

14. The method of claim 13, wherein the adjusting the frequency of the radio frequency emitted based upon the determined attenuation further includes:
comparing the determined attenuation with a predefined range of attenuation data ranges, wherein each predefined range of attenuation data ranges are associated with a frequency.

15. The method of claim 10, further comprising performing thermoacoustic imaging of the region of interest using the tuned RF applicator.

16. The method of claim 15, further comprising determining one or more parameters of the object of interest from the thermoacoustic imaging.

17. The method of claim 16, wherein the one or more parameters of the object of interest comprise at least one of fractional fat content and temperature.

18. The method of claim 17, wherein the region of interest comprises tissue and further wherein the object of interest comprises at least one reference, and wherein the at least one reference and the object of interest are different types of tissue.

19. The method of claim 18, wherein the different types of tissue are selected from a group consisting of muscle tissue, fat tissue, blood vessel tissue, liver tissue, and kidney tissue.

20. A system for enhancing radio frequency energy delivery to a region of interest comprising an object of interest and a reference that are separated by at least one boundary, the system comprising:
a thermoacoustic imaging system comprising a tunable radio frequency (RF) applicator which includes a waveguide and a feed probe configured to emit RF energy pulses into the region of interest to heat the object of interest and the reference that are separated by at least one boundary, means to adjust a direct-current between the waveguide and the feed probe, and an acoustic receiver configured to receive multipolar acoustic pressure waves generated in response to the heating of the object of interest and the reference that are separated by at least one boundary, wherein the multipolar acoustic pressure waves are generated in the region of interest at the boundary; and
one or more processors;
one or more memories storing program instructions for an application executable on the one or more processors to:
determine a peak-to-peak amplitude of at least one multipolar acoustic pressure wave;
determine an updated tuning setting for the RF applicator based upon the determined peak-to-peak amplitude;
cause the RF applicator to emit a RF energy pulse at the determined updated tuning settings.

21. The system of claim 20, wherein, to determine an updated tuning setting for the RF applicator based upon the determined peak-to-peak amplitude, the application is further executable on the one or more processors to:
determine a preferential peak-to-peak amplitudes in the region of interest; and
determine the tuning setting for the RF applicator based upon the determined peak-to-peak amplitude and the preferential peak-to-peak amplitude.

22. The system of claim 20, wherein, the application is further executable on the one or more processors to:
determine one or more parameters of the object of interest based upon received multipolar acoustic pressure waves from the region of interest caused by the RF applicator emitting RF energy at the determined updated tuning settings.

23. The system of claim 22, wherein the one or more parameters of the object of interest are at least one of fractional fat content and temperature.

24. The system of claim 23, wherein the region of interest comprises tissue and further wherein the object of interest and the at least one reference are different types of tissue.

25. The system of claim 24, wherein the different types of tissue are selected from a group consisting of muscle tissue, fat tissue, blood vessel tissue, liver tissue, and kidney tissue.

26. A system for enhancing radio frequency energy delivery to a region of interest comprising an object of interest and a reference that are separated by at least one boundary, the system comprising:
a thermoacoustic imaging system comprising a tunable radio frequency (RF) applicator which includes a waveguide and a feed probe configured to emit RF energy pulses into the region of interest to heat the object of interest and the reference that are separated by at least one boundary, means to adjust a direct-current between the waveguide and the feed probe, and an acoustic receiver configured to receive multipolar acoustic pressure waves generated in response to the heating of the object of interest and the reference that are separated by at least one boundary, wherein the multipolar acoustic pressure waves are generated in the region of interest at the boundary; and
one or more processors;
one or more memories storing program instructions for an application executable on the one or more processors to:
cause the RF applicator to emit one or more RF energy pulses into the region of interest;
detect with an acoustic receiver, at least one multipolar acoustic pressure wave generated in the region of interest in response to the emitted one or more RF energy pulses;
process the detected multipolar acoustic pressure wave to determine a peak-to-peak amplitude thereof;
identify a tissue type of the region of interest based upon the determined peak-to-peak amplitude;
compare the determined peak-to-peak amplitude with an expected peak-to-peak amplitude based upon the identified tissue type to determine attenuation of the RF signal by the region of interest;
adjust a frequency of the radio frequency emitted by the RF applicator based upon the determined attenuation; and
cause the RF applicator to emit one or more RF energy pulses into the region of interest at the adjusted frequency.

27. The system of claim 26, wherein, to adjust a frequency of the radio frequency emitted by the RF applicator based upon the determined attenuation, the application is further executable on the one or more processors to:
compare the determined attenuation with a predefined range of attenuation data ranges, wherein each predefined range of attenuation data ranges are associated with a frequency.

28. The system of claim 26, wherein the identify the tissue type of the region of interest is determined by comparing the determined peak-to-peak amplitude with a table of predefined data ranges, each data range associated with a tissue type.

29. The system of claim 28, wherein each tissue type is associated with an expected peak-to-peak amplitude.

30. The system of claim 29, wherein, to adjust a frequency of the radio frequency emitted by the RF applicator based upon the determined attenuation, the application is further executable on the one or more processors to:
compare the determined attenuation with a predefined range of attenuation data ranges, wherein each predefined range of attenuation data ranges are associated with a frequency.
